(19) United States
(12) Patent Application Publication
(10) Pub. No.: US 2021/0302402 A1
(43) Pub. Date: Sep. 30, 2021

(54) Mobile AI, Cantilever, Robot and Drone Applications

(71) Applicant: Keith DeSanto, St Petersburg, FL (US)

(72) Inventor: Keith DeSanto, St Petersburg, FL (US)

(21) Appl. No.: 16/828,832

(22) Filed: Mar. 24, 2020

(51) Int. Cl. G01N 33/00; G06N 20/00; G06N 5/04; G05D 1/00; G01N 33/24

(52) U.S. Cl. CPC G01N 33/0098; G06N 20/00; G06N 5/04; G01N 2033/245; G01N 33/0036; G01N 33/24; G05D 1/0094

(57) ABSTRACT

Robots and Drones using cantilevers combined with artificial intelligence, machine learning and data mining to detect pathogens in the agriculture industry.

Unmanned land, air or sea mobile vehicles (Robots and Drones) using cantilevers combined with artificial intelligence, machine learning and data mining to detect pathogens in the agriculture industry.

Pathogens in the agriculture are currently detected by other applications that require time, higher costs and expensive technology. Our new AI platforms are based on Mobile Cantilever nanotechnology, data mining through high-definition pictures and machine learning algorithms known as “MCology™”. The AI platform learns about plant and crop pathogens, how to detect them and how to negate pathogens on a single plant or an entire crop. Our new invention has begun to anticipate and forecast threats to plants, bushes, trees and entire crops.

Our new disease, virus, fungus, contamination, air quality/pollution level detection process uses numerous algorithms. Cantilevers can calculate weight of pathogens in femtograms, determine level of air pollution while our AI platform can determine mass of pathogen, contamination type, and what particles are in the air. Our new MCology™ platform can detect many different points of data such as what toxin or pathogen is affecting plants, trees, bushes or entire crops. MCology™ detects fungus, diseases, viruses, and contamination type, particular molecules, the stage of the pathogen and how to control it using our AI, data mining and machine learning algorithms. If a certain bacterium is causing disease, we can determine which bacteria is it. This new invention can detect how long the bacteria has been affecting the plant by level of colonies and size of the bacterium. This new invention can also at times determine where the bacteria came from such as waste water run-off, pests, agricultural machinery, air pollution or by weather events. The new invention can also determine how long the issue has been affecting the plant by algorithms that calculate and learn the duration of the issue using the machine learning. Each time an event has occurred, the AI platform learns from that event.

Daily rapid sampling using our invention can also predict what kind if outcome will occur in the field of agriculture crops.

The present invention can measure weight and size of biological and or chemical elements by use an unmanned drone, robot, land, air or sea roving mobile vehicle equipped with an array of cantilevers that relay data to an AI platform. The drones and robots can be powered by solar energy, nuclear energy, battery or combustion engine and are unmanned. The vehicles can be operated by wireless technology, cell phones or via satellite. The size and weight of the unmanned vehicles ranges from large multi-ton to nanotechnology sized vehicles. With this new invention, we can detect the signature of pathogens with ease. With our new detection system, we are able to immediately ascertain the type of pathogen, particular molecules, the stage of the pathogen and how to control it using AI data mining.

Disease in plants, food and water including spreading of pathogens from environmental contamination contact can be controlled if an alert system is employed and an assessment is made immediately. Our application is just that system. Our instantaneous Rapid detection of virulence can be completed every minute, every day or every month. Our platform can be used for agricultural applications indoor and outdoor. MCology is also an early warning system of pathogens in the food, water and agricultural industries before they are sold on the store shelves.

Cantilever Technology. Cantilevers utilizing nanotechnology have been in use since early 2000. A cantilever can be described as a small diving board. The board is anchored (anchored end) at one end and free to fluctuate at the other end (non-anchored end). Cantilevers can be perpendicular to a flat, vertical or slanted surface and are rigid structures. They be made of any structural element such as a thin wire made out of silicon. If the cantilever is subjected to a load of any type of weight, it will bend. The stress at the non-anchored end from the load can be weighed by the angle of the board using different lengths. Our mobile cantilever technology utilizes stacked arrays of many cantilevers made of glass, silicone, and metal.

Current cantilever technology uses static applications that are limited in process. These applications take time to retrieve the data from the cantilevers and then proceed with analyzing the data that may be inaccurate. This procedure is time consuming and costly. Rapid detection and analysis from our mobile drones and robots can produce data immediately. Our Mobile Cantilever Technology can detect the full array of pathogens using AI algorithms immediately.

One component of our application of pathogen and molecular detection is done by size. Our technology can detect sizes of particles in femtograms. A femtogram is a measurement of weight where a femtogram is equal to 0.000 000 000 000 001 grams.

Our AI system is able to utilize datamining collected from the cantilevers by learning the structure, weight, mass size, behavior, fingerprints and footprints of pathogenic material.

Robots, drones and cantilevers through Artificial Intelligence “AI” platforms are replacing workers with new technology and machine learning algorithms. Indoor environments are benefiting from this new technology by learning about indoor environments, their cleanliness, the causes of un-sanitary conditions and the sanitizing of the indoor space. Monitoring an indoor environment such as a nursery costly and time consuming. Our application is quick, cost efficient and can be deployed with ease.

Natural intelligence is demonstrated by humans and animals. Artificial Intelligence “AI” also referred to as “machine intelligence” is non-human intelligence exhibited by machines. AI machines are also called intelligent agents. Machine Learning is the study and implementation of algorithms and statistical models that computers “learn” from. The computers set out to use data mining (pattern discovery) to perform specific tasks. The learning can be in the form of patterns, interpretations and presumptions using data mining to arrive at conclusions. High definition pictures, video, statistics among other data are also used to extract information from the complied data to detect levels of toxins and pathogens and forecast future events. For the sake of this invention, Artificial Intelligence, machine learning and data mining should be considered working together to form our AI platform.

A robot is defined as a machine that is programmable and can carry out a series of tasks. A robot that is programmed and managed by an AI machine learning machine platform is a “Robot Learning Machine” Our Robots have cantilevers on or within the body of the robot.

A drone or a “UAV” unmanned ariel vehicle is defined as a pioletless flying robot. Our drones have cantilevers on or within the body of the drone.

Example 1. Drones, cantilevers and AI in the agricultural industry. One of the biggest challenges in all agricultural industries including the hemp and cannabis industries is to contain and avoid disease, fungus and viruses. An early warning system and information about how to correct or avoid the threat will not only save money from crop destruction but our new system may save businesses from going under.

An example would be that our system would learn what caused destruction in specific times with specific crops such as in the cannabis industry. At a grove in California, most of the cannabis plants would not flower and no one knew why. Our system learned that a cannabis canker was the culprit long before the problem was known. Our system also learned how to manage the problem by leaning when to apply agricultural products such as rhamnolipid, how much was needed and what ratio of mono to di-rhamnolipid was effective.

Example 2. Robots, cantilevers and AI in the agricultural industry. In order to take high definition pictures for our data mining algorithms, drones must be very still in order to get 360 degrees and to avoid blurry pictures. Our robots can affix themselves to the walls, grounds or erect a quick platform where wind and or bright light are either needed or avoided depending on what the machine learning algorithm has learned. An example of this be during the rainy season where wind and rain pose a problem for cantilever reading particles as well obtaining high definition pictures. The quick setup of a mobile enclosure negates these problems. The AI platform would manage all aspects of deploying such a cover through the robot. A simple example of our enclosure would be similar to a spring-loaded tent that is big enough to cover a branch or a plant and shield it from weather elements that can just as easily be deconstructed in seconds with very little effort of behalf of the AI platform.

Example 3. Our learning platform has also learned how drones can affix themselves to plants and trees or just rest on single branches of trees or several thin branches to gather data. Algorithmic calculations determine the weight the branch or leaf can withstand by measuring the thickness of branch or cluster of leaves by drone mapping. An example of this would be if is noon and in the middle of the summer where the temperature is over 80 degrees and sun is directly overhead. When working with trees such as the african palm tree that can grow be over 20 meters, the drone would not only need shade to take some high definition pictures underneath the palm leaf, but need to sample the surrounding air. Resting on a leaf with shade above and enough drag coefficient on the surface of the leaf to not get blown about by a gust of wind. Cantilevers can be programmed to calculate wind speed. The AI platform not only learns from its prior mistakes, but also learns to take less and less time to complete each task. In one instance, our machine learning algorithm was able to not set the drone down because of a large snake was occupying the branch.

Example 4. One of our drones' purpose in the agricultural industry is to survey and calculate problematic areas and determine sanitary or unsanitary conditions of an indoor or outdoor, open or closed structure housing agricultural products. Our drones compile the following data. They map out the area of the grove, nursery or farm that needs to be scanned. If the structure is closed like that of a nursery, the AI platform directs the drones and robots to work together to map out the internal items that are located inside the structure by using specific algorithms. The combined data of structure area and internal contents will form the “shell” and be transferred into the AI platform. Then, mapping software of the indoor structure will use visual, laser mapping, and grid calculation software whereas the entire area including the internal contents of the structure will form a three-dimensional schematic view with x, y and z data points. This data will be calculated and transferred into our AI platform. Patterns of soil collection and its moisture will be transferred into the AI platform to see if the soil collection is arid or not and to what degree the aridity is. The irrigation system in the crop area can be revised to add or limit water. Our AI platform can calculate a change in water distribution flow or a problem with the entire irrigation system. An example of this would be that after building a database of irrigation success and failures, data mining has learned about other facilities various irrigation systems and which ones had more success in utilizing less water.

Example 5. Gathering data without tainting the data. Using Robots and Drones will limit error that humans are prone to making. Humans tend to transfer pathogens on their clothes and soles of shoes. These infection metrics can be negated. They also tend to make mistakes in harvest calculations. Using AI to map the area of the grove will also improve the quality and quantity of the crop harvest. An example of this is by using an AI algorithm to pre-determine if the crop is viable, valuable and can be easily harvested. By this, high definition pictures through data mining, such as footprints and finger prints of healthy cell structures builds learning from past harvests. Through this, our platform can forecast some crop events such as what level of health is the plant at the time of harvest.

Example 6. Our AI platform of algorithms learns about certain hazards such as over and under fertilizing, over and under watering and certain weather events that are either positive or have a negative effect. An example would be when a toxic chemical is emitted into the air or soil by accident, our cantilevers will detect immediately what the toxin is where the problem started. Our AI platform learns how to correct those problems.

Example 7. During many agricultural applications, pathogens can be introduced into greenhouses, nurseries and outdoor crops through machinery and tools used in the fields. Our AI algorithm scans and tests for toxins and pathogens before the item in used in the field or on crops. Machine learning learns how to avoid these problems by understanding how and when a toxin or pathogen is introduced to the plant.

1. An application using machine learning, data mining and artificial intelligence combined with one or more cantilevers where the cantilever is imbedded in a drone or robot to detect fungus, disease and virus causing pathogens in the agricultural industry.

2. An application as in claim 1 where the robot or drone operates on land, in the air or in a body of water.

3. An application as in claims 1 and 2 where the robot or drone can detect fungus, diseases and viruses in soil.

4. An application as in claims 1 and 2 where the done or robot can detect contamination or air pollution particles.

5. An application as in claims 1 through 4 where our machine learning and data mining algorithms with data from cantilevers can detect specific pathogens by weight in femtograms combined with high definition pictures.

6. An application as in claims 1 through 5 where the application utilizes arrays of cantilevers which can be stacked or placed on or inside the robot or drone.

7. An application as in claims 1 through 6 where the application utilizes a self-cleaning system, or a reusable cartridge or a disposable cartridge.

8. An application as in claims 1 through 7 where the data obtained can be used to forecast crop harvests, crop events, and level of health or sickness in crops.
